(19) United States
(12) Patent Application Publication
(10) Pub. No.: US 2006/0063161 A1
(43) Pub. Date: Mar. 23, 2006

(54) Detecting RNAi using SELDI mass spectrometry

(75) Inventors: Gurucharan Reddy, Fremont, CA (US); William E. Rich, Redwood Shores, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Ciphergen Biosystems, Inc., Fremont, CA (US)

(21) Appl. No.: 10/948,028

(22) Filed: Sep. 22, 2004

(51) Int. Cl. C12Q 1/68; C07H 21/02

(52) U.S. Cl. 435/6; 536/23.1

(57) ABSTRACT

The present invention relates to the fields of protein expression and molecular biology. In particular, the present invention includes methods for monitoring the levels of polypeptide inhibition caused by inhibitory nucleic acids.

### FIELD OF THE INVENTION

The present invention relates to the fields of protein expression and molecular biology. In particular, the present invention includes methods for monitoring the levels of polypeptide inhibition caused by inhibitory nucleic acids.

### BACKGROUND OF THE INVENTION

Cell function, both normal and pathologic, depends, in large part, on the proteins expressed by the cell. Powerful tools for analyzing the function of proteins are nucleic acids that inhibit protein expression by interfering with mRNA prosessing, particularly the translation process. These tools include antisense nucleotides, ribozymes and siRNAs. By interfering with mRNA processing before it can be translated to protein, inhibitory nucleic acids allow for the creation of gene-specific loss-of-function mutations, either transiently or permanently using simpler techniques than those employed when creating genomic knockout mutants.

Analysis of protein function requires tools that can resolve the complex mixture of molecules in a cell, quantify them and identify them, even when present in trace amounts. To facilitate analysis, sensitivity of the detection method needs to be maximized. One means for increasing sensitivity and accuracy of the detection method is to minimize manipulation of the sample prior to the actual detection step.

One popular method is gel electrophoresis. Frequently, a first separation of proteins by isoelectric focusing in a gel is coupled with a second separation by sodium dodecyl sulfate-polyacrylamide gel electrophoresis (SDS-PAGE). The result is a map that resolves proteins according to the dimensions of isoelectric point (net charge) and size (i.e., mass). However useful, this method is limited in several ways. First, the resolution power in each of the dimensions is limited by the resolving power of the gel. For example, molecules whose mass differ by less than about 5% or less than about 0.5 pI are often difficult to resolve. Second, gels have limited loading capacity, and thus sensitivity; one may not be able to detect biomolecules that are expressed in small quantities. Third, small proteins and peptides with a molecular mass below about 10-20 kDa are not observed. Fourth, for cell lysates, the lysate frequently requires some form of fractionation prior to gel analysis to simplify the molecular mixture.

Other analytical methods may overcome one or more of these limitations, but they are difficult to combine efficiently. For example, analytical chromatography can separate biomolecules based on a variety of analyte/adsorbent interactions, but multi-dimensional analysis is difficult and time consuming. Furthermore, the methods are limited in typically limited in sensitivity.

### SUMMARY OF THE INVENTION

To overcome the limitations noted above, the present invention provides a method of analyzing the inhibition of polypeptide expression or function comprising (a) expressing polypeptides in a first translation system, where the first translation system includes at least one inhibitory nucleic acid directed toward at least one mRNA, followed by (b) measuring expression of at least one polypeptide in the translation system by affinity mass spectometry. The translation system may be an in vitro or in vivo system, depending on convenience and the polypeptides being analyzed. In one aspect of the method, the translation system is in contact with an affinity surface of an affinity mass spectrometry probe, where the affinity surface captures polypeptides expressed by the expression system in situ. In another aspect, the inhibitory nucleic acid used in the method may be an antisense nucleic acid, a ribozyme and an siRNA. In a preferred aspect, the inhibitory nucleic acid is an siRNA comprising between 18 and 25 paired bases.

Generally, least one polypeptide is inhibited by the at least one inhibitory nucleic acid. In some aspects of the above method, at least one inhibitory nucleic acid in the translation system inhibits expression of a cell receptor selected from a nuclear receptor, a cytoplasmic receptor and a cell surface receptor. In other aspects at least one of the polypeptides is a ligand for the cell receptor.

The above method also may have an mRNA that encodes a component of a signaling pathway. In these situations, at least one of the polypeptides is a component of the pathway. In some aspects the mRNA encodes a kinase or a phosphatase. In variations of these aspects at least one of the polypeptides is a substrate for the kinase or phosphatase.

In still other aspects, the mRNA of the above method encodes a protease, with variations of these aspects where at least one of the polypeptides is a substrate for the protease.

The method may also have at least one polypeptide that is a polypeptide that interacts with a polypeptide encoded by the mRNA.

In all of the variations to the method noted above, affinity mass spectrometry may comprise capturing the at least one polypeptide on a mass spectrometry probe comprising a surface having a chromatographic capture reagent attached thereto. Alternatively, affinity mass spectrometry comprises capturing the at least one polypeptide on a mass spectrometry probe comprising a surface having a biospecific capture reagent attached thereto, wherein the biospecific capture reagent binds the polypeptide, or affinity mass spectrometry further comprises SEND.

In certain aspects, the method above also includes (c) expressing second polypeptides in a second translation system, where the expression system does not comprise the at least one siRNA; (d) measuring expression of at least one of the second polypeptides in the expression system by affinity mass spectrometry; and, (e) comparing expression of the polypeptides in the first and second expression systems. An alternative to these aspects involves performing steps (a) and (b) on a plurality of first expression systems and measuring a plurality of first polypeptides; performing steps (c) and (d) on a plurality of second expression systems measuring a plurality of second polypeptides, where comparing the polypeptides comprises using the measurements as a learning set to train a learning algorithm, thereby generating a classification model, wherein the classification model uses measurement of at least one polypeptide to classify an unknown sample as belonging to the first or second expression system.

The present invention also includes kits that combine reagents and apparatus useful in performing the methods of the invention, One such kit comprises a first expression system capable of expressing at least one inhibitory nucleic acid; and at least one affinity mass spectrometry probe. This kit may also include instructions to use the kit to detect expression of a polypeptide whose expression is inhibited by the inhibitory nucleic acid. Another option is to include a second expression system that does not express the inhibitory nucleic acid. This latter option may also include instructions to compare expression of a polypeptide in both the first and second expression systems.

A second kit embodiment has at least one inhibitory nucleic acid capable of inhibiting expression of at least one expressed protein of a species; and, at least one affinity mass spectrometry probe capable of binding at least one of the expressed proteins.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

An “inhibitory nucleic acid” is a nucleic acid that prevents normal expression levels of a protein in a sequence-specific manner, e.g., an antisense nucleic acid, ribozyme or siRNA.

“Translation system” is any liquid system where nucleic acids are recognized as templates encoding polypeptides and are translated to the corresponding peptide according to a genetic code.

“Expressed protein of a species” refers to an expressed protein found in a particular species of the plant or animal kingdoms.

“Biochemical substrate” refers to a molecule that is recognized and modified by an enzyme formed at least partially from a polypeptide, nucleic acid, polysaccharide or a combination of any of these biomolecules.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm2 to about 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Probe” refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” (“SEAC”) or “affinity gas phase ion spectrometry” (e.g., “affinity mass spectrometry”) is a version of the SELDI method that uses a probe comprising an absorbent surface (a “SEAC probe”). “Adsorbent surface” refers to a sample presenting surface of a probe to which an adsorbent (also called a “capture reagent” or an “affinity reagent”) is attached. An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates (e.g., Cu, Fe, Ni), hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. An adsorbent is “bioselective” for an analyte if it binds the analyte with an affinity of at least 10-8M, 10-9M, 10-10M, 10-11 M or 10-12M. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

In a preferred embodiment affinity mass spectrometry involves applying a liquid sample comprising an analyte to the adsorbent surface of a SELDI probe. Analytes, such as polypeptides, having affinity for the adsorbent bind to the probe surface. Typically, the surface is then washed to remove unbound molecules, and leaving retained molecules. The extent of analyte retention is a function of the stringency of the wash used. An energy absorbing material (e.g., matrix) is then applied to the adsorbent surface. Retained molecules are then detected by laser desorption/ionization mass spectrometry.

SELDI is useful for protein profiling, in which proteins in a sample are detected using one or several different SELDI surfaces. In turn, protein profiling is useful for difference mapping, in which the protein profiles of different samples are compared to detect differences in protein expression between the samples.

“Surface-Enhanced Neat Desorption” or “SEND” is a version of SELDI that involves the use of probes (“SEND probe”) comprising a layer of energy absorbing molecules attached to the probe surface. Attachment can be, for example, by covalent or non-covalent chemical bonds. Unlike traditional MALDI, the analyte in SEND is not required to be trapped within a crystalline matrix of energy absorbing molecules for desorption/ionization. “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-methoxy)silyl propyl methacrylate. In another embodiment, the composition is a co-polymer comprising α-cyano-4-methacryloyloxycinnamic acid and octadecylmethacrylate (“C18 SEND”). SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

SEAC/SEND is a version of SELDI in which both a capture reagent and an energy absorbing molecule are attached to the sample presenting surface. SEAC/SEND probes therefore allow the capture of analytes through affinity capture and desorption without the need to apply external matrix. The C18 SEND biochip is a version of SEAC/SEND, comprising a C18 moiety which functions as a capture reagent, and a CHCA moiety which functions as an energy absorbing moiety.

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Solid support” refers to a solid material which can be derivatized with, or otherwise attached to, a chemical moiety, such as a capture reagent, a reactive moiety or an energy absorbing species. Exemplary solid supports include, without limitation, chips (e.g., probes), microtiter plates, membranes and chromatographic resins.

“Chip” refers to a solid support having a generally planar surface to which a chemical moiety may be attached. Chips that are adapted to engage a probe interface are also called “probes.”

“Biochip” refers to a chip to which a chemical moiety is attached. Frequently, the surface of the biochip comprises a plurality of addressable locations, each of which location has the chemical moiety attached there.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, Q-10, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, Q-10, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 and Q-10 biochips have quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu⁺⁺ and Ni⁺⁺, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); U.S. Pat. No. 6,555,813 (Beecher et al., “Sample Holder with Hydrophobic Coating for Gas Phase Mass Spectrometer,” Apr. 29, 2003); U.S. patent application U.S. 2003 0032043 A1 (Pohl and Papanu, “Latex Based Adsorbent Chip,” Jul. 16, 2002) and International patent application WO 03/040700 (Um et al., “Hydrophobic Surface Chip,” May 15, 2003); U.S. patent application 60/367,837, (Boschetti et al., “Biochips With Surfaces Coated With Polysaccharide-Based Hydrogels,” May 5, 2002) and U.S. patent application entitled “Photocrosslinked Hydrogel Surface Coatings” (Huang et al., filed Feb. 21, 2003).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Data generation in mass spectrometry begins with the detection of ions by an ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes that are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002 0193950 A1 (Gavin et al., “Method or analyzing mass spectra,” Dec. 19, 2002); U.S. 2003 0004402 A1 (Hitt et al., “Process for discriminating between biological states based on hidden patterns from biological data,” Jan. 2, 2003); and U.S. 2003 0055615 A1 (Zhang and Zhang, “Systems and methods for processing biological expression data” Mar. 20, 2003).

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

### DETAILED DESCRIPTION

I. Introduction

The present invention provides methods for determining the presence and degree of inhibition to polypeptide expression caused by inhibitory nucleic acids. The inhibitory nucleic acids described herein may be introduced to cells alone or in combination with other reagents as required by the particular application. The inhibitory nucleic acids may be transiently or stably expressed in cells, or may be admixed in cell-free translation systems, or used as naked nucleic acids to transform cells or tissues.

Polypeptides whose expression levels are determined using the methods of the invention need not be inhibited by the inhibitory nucleotides described, but rather have their expression levels modified through an increase or decrease in expression. This modification of determined polypeptide expression level is however a consequence of the effect of an inhibitory nucleic acid on the expression of at least one polypeptide within the same translation system as the polypeptide expression being determined.

More particularly, the methods of the invention generally involve using an inhibitory RNA to inhibit expression of an mRNA in a translation system, and then using a high throughput proteomics system, such as SELDI, to detect levels of expression of one or more different proteins in the system. Then, one can compare the changes in protein expression to identify proteins whose expression has changed. This can include a polypeptide encoded by the inhibited mRNA, itself, as well as any other polypeptide, the expression of which may be modified as a result of inhibition of the inhibited mRNA. This can occur when the inhibited mRNA encodes a polypeptide that has an action on another polypeptide. Such instances include, for example, when the inhibited mRNA encodes an enzyme (such as a phosphorylase, kinase or a protease), a component in a signaling pathway (such as a receptor), or a modifier of gene expression.

A variety of methods for determining polypeptide expression levels are provided, the preferred method being gas-phase ion spectrophotometry.

II. Inhibitory Nucleic Acids

Inhibitory nucleic acids of the present invention are molecules inhibiting polypeptide expression in a translation system. Exemplary nucleic acids include antisense molecules, ribozymes and siRNA, each of which is described in more detail below.

A. Types of Inhibitory Nucleic Acids

1. Antisense Nucleic Acids

Antisense nucleic acids are nucleic acids complementary to a target RNA, preferably an mRNA, and inhibit protein synthesis by interacting with the target RNA. (See, for example, U.S. Pat. Nos. 5,718,709; 5,610,288; 5,801,154; 5,789,573; 5,739,119 and 5,759,829). Typical antisense embodiments of the present invention will inhibit polypeptide expression in a translation system by at least 10%, preferably at least 20% more preferably at least 30, 40, 50, 60, 70, 80 or 90%, and ideally by 100%.

In one embodiment, the antisense nucleic acids comprise DNA or derivatives thereof. In another embodiment, the nucleic acids comprise RNA or derivatives thereof. In a third embodiment, the nucleic acids are modified DNAs as described below. In a fourth embodiment, the nucleic acid sequences comprise peptide nucleic acids or derivatives thereof. In each case, preferred compositions comprise a sequence region that is complementary, and more preferably substantially-complementary, and even more preferably, completely complementary to the target RNA. Suitable modifications of antisense nucleic acids are known in the art and discussed in detail herein.

Antisense nucleic acids of the invention may be delivered to cells using any method known to those of skill in the art. In addition to delivery methods discussed below, the use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen. It has been demonstrated that several molecules of the MPG peptide coat the antisense nucleic acids and can be delivered into cultured mammalian cells in less than 1 hour with relatively high efficiency (90%). Further, the interaction with MPG strongly increases both the stability of the nucleic acid to nuclease and the ability to cross the plasma membrane.

2. siRNA

siRNA molecules are small double-stranded RNAs that elicit a form of sequence-specific gene inactivation. Zamore, Phillip et al., Cell, 101:25-33 (2000). SiRNAs are preferably between 16 and 25, more preferably 17 and 23 and most preferably between 18 and 20 base pairs long, each strand of which has a 3′ overhang of 2 or more nucleotides. Functionally, the sequence-specific gene inactivation elicited by siRNAs is termed “RNA interference”. RNA interference has been shown to exist in mammalian cell lines, oocytes, early embryos and some cell types (see e.g., Elbashir, Sayda M., et al., Nature, 411:494-497 (2001)). For a review of RNAi and siRNA expression, see Hammond, Scott M et al., Nature Genetics Reviews, 2:110-119; Fire, Andrew, TIG, 15(9):358-363 (1999); Bass, Brenda L., Cell, 101:235-238 (2000).

SiRNAs may be introduced to a cell using any means known in the art. Several methods, including gene therapy techniques for introduction of nucleic acids into a cell are detailed below. For example, in addition to transient transfection using si-RNA-producing vectors, siRNA molecules may be introduced into chromosomal DNA of a cell, producing a cell stably expressing a cell. Methods for producing stably expressing cells are known to those of skill in the art, and include homologous recombination techniques. Introducing an siRNA into a cell causes the RNA targeted by the siRNA to be destroyed (Sharp et al., Genes Dev. 13:139, 1999; Wianny et al., Nat. Cell Biol. 2:70, 2000). In certain embodiments of this invention, a control element driving transcription in the target cell is operably linked to a coding sequence for an siRNA specifically directed to the target RNA.

3. Ribozymes

The present invention also contemplates inhibitory nucleic acids having nuclease activity, such as ribozymes. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an nucleic acid substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction. For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990).

Six basic varieties of naturally-occurring enzymatic RNAs are known presently. Each can catalyze the hydrolysis of RNA phosphodiester bonds in trans (and thus can cleave other RNA molecules) under physiological conditions.

The enzymatic nucleic acid molecule may be formed in a hammerhead, hairpin, a hepatitis δ-virus, group I intron or RNaseP RNA (in association with an RNA guide sequence) or Neurospora VS RNA motif. Examples of hammerhead motifs are described by Rossi et al (1992). Examples of hairpin motifs are described by Hampel et al. (Eur. Pat. Appl. Publ. No. EP 0360257), Hampel and Tritz (1989), Hampel et al. (1990) and U.S. Pat. No. 5,631,359. An example of the hepatitis 6 virus motif is described by Perrotta and Been (1992); an example of the RNaseP motif is described by Guerrier-Takada et al. (1983); Neurospora VS RNA ribozyme motif is described by Collins (Saville and Collins, 1990; Saville and Collins, 1991; Collins and Olive, 1993); and an example of the Group I intron is described in (U.S. Pat. No. 4,987,071). All of these species are contemplated as part of the present invention, with the substitution of an appropriate nucleotide sequence at the internal guide sequence site. For purposes of the present invention, appropriate nucleotide sequences include any nucleotide sequence having 85%, more preferably at least 90%, most preferably at least 95% and ideally at least 100% identity to a nucleotide sequence complementary to the target RNA Thus the ribozyme constructs need not be limited to specific motifs mentioned herein.

Small enzymatic nucleic acid motifs (e.g., of the hammerhead or the hairpin structure) may be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g., Scanlon et al., 1991; Kashani-Sabet et al., 1992; Dropulic et al., 1992; Weerasinghe et al., 1991; Ojwang et al., 1992; Chen et al., 1992; Sarver et al., 1990). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference; Ohkawa et al., 1992; Taira et al., 1991; and Ventura et al., 1993). In addition, ribozymes of the invention may be delivered to cells using techniques for delivering any nucleic acid to cells commonly known in the art, preferred methods of which are detailed below.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perrault et al., 1990; Pieken et al., 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements. B. Synthesizing Nucleic acids

Nucleic acids of the present invention may be constructed using any suitable method known to one of skill in the art. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994).

Nucleic acids may be chemically synthesized according to the solid phase phosphoramidite triester method first described by Beaucage & Caruthers, Tetrahedron Letts., 22:1859-1862 (1981), using an automated synthesizer, as described in Van Devanter et. al., Nucleic Acids Res., 12:6159-6168 (1984). Purification of nucleic acids is by either native acrylamide gel electrophoresis or by anion-exchange HPLC as described in Pearson & Reanier, J. Chrom., 255:137-149 (1983).

Where desirable, one of skill in the art will recognize many ways of generating alterations in a given nucleic acid sequence. Such well-known methods include site-specific mutagenesis, PCR amplification using degenerate nucleic acids, exposure of cells containing the nucleic acid to mutagenic agents or radiation, chemical synthesis of a desired nucleic acid (e.g., in conjunction with ligation and/or cloning to generate large nucleic acids) and other well-known techniques. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991).

In some embodiments, modified bases are incorporated into nucleotides of the invention. Instances where incorporating modified bases may be desirable include applications where it is advantageous to extend the half-life of the nucleic acid, or where the modification facilitates cell entry. Synthetic procedures are altered as needed according to known procedures, particularly if modified phosphodiester linkages are used. In this regard, Uhhnann, et al. (1990, Chemical Reviews 90: 543-584) provide references and outline procedures for making nucleic acids with modified bases and modified phosphodiester linkages.

Preferred nucleotide analogs are unmodified G, A, T, C and U nucleotides; pyrimidine analogs with lower alkyl, alkynyl or alkenyl groups in the 5 position of the base and purine analogs with similar groups in the 7 or 8 position of the base. Other preferred nucleotide analogs are 5-methylcytosine, 5-methyluracil, diaminopurine, and nucleotides with a 2′-O-methylribose moiety in place of ribose or deoxyribose. As used herein lower alkyl, lower alkynyl and lower alkenyl contain from 1 to 6 carbon atoms and can be straight chain or branched. These groups include methyl, ethyl, propyl, isopropyl, butyl, isobutyl, tertiary butyl, amyl, hexyl and the like. A preferred alkyl group is methyl.

Nulciec acids of the invention may include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, olate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference.

For example, covalent linkage of a cholesterol moiety to a nucleic acid can improve cellular uptake by 5- to 10-fold which in turn improves DNA binding by about 10-fold (Boutorin et al., 1989, FEBS Letters 254: 129-132). Ligands for cellular receptors may also have utility for improving cellular uptake, including, e.g. insulin, transferrin and others. Similarly, derivatization of oligonucleotides with poly-L-lysine can aid nucleic acid uptake by cells (Schell, 1974, Biochem. Biophys. Acta 340: 323, and Lemaitre, et al., 1987, Proc. Natl. Acad. Sci. USA 84: 648).

Certain protein carriers can also facilitate cellular uptake of nucleic acids, including, for example, serum albumin, nuclear proteins possessing signals for transport to the nucleus, and viral or bacterial proteins capable of cell membrane penetration. Accordingly, the present invention contemplates derivatization of the subject oligonucleotides with groups capable of facilitating cellular uptake, including hydrocarbons and non-polar groups, cholesterol, poly-L-lysine and proteins, as well as other aryl or steroid groups and polycations having analogous beneficial effects, such as phenyl or naphthyl groups, quinoline, anthracene or phenanthracene groups, fatty acids, fatty alcohols and sesquiterpenes, diterpenes and steroids.

Nucleic acid sizes are given in either kilobases (Kb) or base pairs (bp). These are estimates derived from agarose or acrylamide gel electrophoresis, from sequenced nucleic acids, or from published DNA sequences.

The sequence of isolated oligonucleotides may be verified after using, e.g., the chain termination method for sequencing double-stranded templates of Wallace et al., Gene, 16:21-26 (1981) or using the chemical degradation method of Maxam and Gilbert (1980) in Grossman and Moldave (eds.) Academic Press, New York, Methods in Enzymology 65:499-560. Sequences of short oligonucleotides can also be analyzed by laser desorption mass spectroscopy or by fast atom bombardment (McNeal, et al., 1982, J. Am. Chem. Soc. 104: 976; Viari, et al., 1987, Biomed. Enciron. Mass Spectrom. 14: 83; Grotjahn et al., 1982, Nuc. Acid Res. 10: 4671). Analogous sequencing methods are available for RNA oligonucleotides.

C. Delivering Inhibitory Nucleic Acids to Cells

Inhibitory nucleic acids and expression vectors of the present invention may be admixed directly to cell lysates and other cell-free translation systems. Inhibition of polypeptide expression in cells using inhibitory nucleic acids of the present invention may be through stable transformation of a cell, but is preferably transient in nature. Inhibitory nucleic acids are intended for parenteral, topical, oral, local or intranasall administration. Where desirable inhibitory nucleic acids will be dissolved in a pharmaceutically acceptable excipient, preferably an aqueous excipient. A variety of aqueous excipients may be used, e.g., water, buffered water, 0.4% saline, 0.3% glycine, and the like, including glycoproteins for enhanced stability, such as albumin, lipoprotein, globulin, etc. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

Determination of an effective amount of inhibitory nucleic acid necessary to inhibit polypeptide expression will vary and depend upon the type of application e.g., whether being applied in vitro or in vivo, to cells or lysates, to cells in culture or whole tissue, etc.

Inhibitory nucleic acids of the invention may be tested using any suitable screening technique known to those of skill in the art. By way of example, inhibition of polypeptide expression may be determined in vitro by measuring the amount of polypeptide produced by cultured cells treated with the inhibitory peptide compared to the amount of polypeptide produced by cells not treated with the inhibitory peptide. In some embodiments, cells may be contacted with varying amounts of inhibitory nucleic acid. For purposes of this invention, a inhibitory nucleic acid is considered an effective inhibitor of polypeptide expression if the amount of polypeptide produced by treated cells is at least 30%, more preferably 40%, still more preferably 50%, more advantageously 60%, most advantageously 75%, ideally by at least 80% less than the amount of polypeptide produced by untreated cells. Polypeptide expression may be measured using any suitable means known in the art.

Within certain aspects of the invention, inhibitory nucleic acids, or vectors expressing them, may be introduced into a host cell utilizing a vehicle, or by various physical methods. Representative examples of such methods include transformation using calcium phosphate precipitation (Dubensky et al., PNAS, 81:7529-7533 (1984)), direct microinjection of such nucleic acid molecules into intact target cells (Acsadi et al., Nature, 352:815-818 (1991)), and electroporation whereby cells suspended in a conducting solution are subjected to an intense electric field in order to transiently polarize the membrane, allowing entry of the nucleic acid molecules.

Direct cellular uptake of inhibitory nucleic acids (whether they are composed of DNA or RNA or both) per se is presently considered a less preferred method of delivery because, in the case of siRNA and antisense molecules, direct administration of inhibitory nucleic acids oligonucleotides carries with it the concomitant problem of attack and digestion by cellular nucleases, such as the RNAses. The mode for administration of the expression cassettes of the present invention takes advantage of known vectors to facilitate the delivery of an expression cassette suitable for expressing the inhibitory nucleic acid in a target cell. Such vectors include plasmids and viruses (such as adenoviruses, retroviruses, and adeno-associated viruses) [and liposomes] and modifications therein (e.g., polylysine-modified adenoviruses [Gao et al., Human Gene Therapy, 4:17-24 (1993)], cationic liposomes [Zhu et al., Science, 261:209-211 (1993)] and modified adeno-associated virus plasmids encased in liposomes [Phillip et al., Mol. Cell. Biol., 14:2411-2418 (1994)].

Other procedures include the use of nucleic acid molecules linked to an inactive adenovirus (Cotton et al., PNAS, 89:6094 (1990)), lipofection (Felgner et al., Proc. Natl. Acad. Sci. USA, 84:7413-7417 (1989)), microprojectile bombardment (Williams et al., PNAS, 88:2726-2730 (1991)), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., Pharmac. Ther., 29:69 (1985); Curiel et al., Proc Natl Acad Sci USA, 88:8850-8854 (1991); Cotten et al., Proc Natl Acad Sci USA, 89:6094-6098 (1992); Curiel et al., Hum Gene Ther, 3:147-154 (1992); Wagner et al., Proc Natl Acad Sci USA, 89:6099-6103 (1992); Michael et al., J Biol Chem, 268:6866-6869 (1993); Curiel et al., Am J Respir Cell Mol Biol, 6:247-252 (1992); Harris et al., Am J Respir Cell Mol Biol, 9:441-447 (1993), and Friedmann et al., Science, 244:1275 (1989)), and DNA ligand (Wu et al., J. of Biol. Chem., 264:16985-16987 (1989)), as well as psoralen inactivated viruses such as AAV or Adenovirus. In one embodiment, the construct is introduced into the host cell using a liposome. Liposome based gene delivery systems are described in Debs and Zhu (1993) WO 93/24640; Mannino and Gould-Fogerite, BioTechniques, 6(7):682-691 (1988); Rose U.S. Pat. No. 5,279,833; Brigham (1991) WO 91/06309; and Felgner et al., Proc. Natl. Acad. Sci. USA, 84:7413-7414 (1987).

D. Labelling Nucleic Acids

The particular label or detectable group used in the assay is not a critical aspect of the invention, as long as it does not significantly interfere with the specific binding of the nucleic acid or antibody used in the assay. In fact, in the preferred detection method, mass spectroscopy, no label is required. When a label is desirable, the detectable group can be any material having a detectable physical or chemical property. Such detectable labels have been well-developed and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g. DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly according to methods well known in the art. As indicated above, a wide variety of labels may be used, with the choice of label depending on sensitivity required, ease of conjugation with the compound, stability requirements, available instrumentation, and disposal provisions.

III. Polypeptide Analysis

Polypeptides suitable for analysis using the methods of the present invention include any polypeptide being expressed from a coding nucleic acid sequence, or otherwise directly or indirectly encoded in an RNA molecule as a necessary course of polypeptide expression, or any polypeptide present in the translation system whose expression may be affected by the presence of an inhibitory nucleic acid.

Polypeptide analysis may be carried out using any suitable method known to those of skill in the art. By way of example, immunological methods such as ELISA assay, spectroscopic, fluoroscopic or calorimetric methods may be used. A preferable method of detection is mass spectrometry, preferably SEND mass spectrometry. Mass spectrometric methods are discussed in more detail, below.

In certain embodiments, polypeptides to be analyzed may be purified partially or to near homogeneity using methods known by those of skill in the art (see, e.g., Scopes, Protein Purification: Principles and Practice (1982)), or, particularly in the case of biochemical substrates, be produced recombinantly (See, e.g., Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel)).

A. Biochip Devices for Capturing Polypeptides

Biochip devices of the claimed invention comprise a biochip having one or more reactive surfaces. Preferably the biochip is suitable for use as a mass spectrometry probe. Where more than one reactive surface is present on the biochip device, each reactive surface should be addressable such that molecules present at each addressable surface may be analyzed independently from molecules present at other surfaces of the biochip. The devices also include a capture reagent that recognizes antibodies, particularly allergen-specific antibodies. The capture reagent may be directly bound to the reactive surface, or may be indirectly bound to the reactive surface through an interfacing linker molecule. Each of these aspects of the invention are sequentially described in greater detail below.

1. Solid Substrates for Biochips

Solid substrates are preferably formed as part of biochips adapted for use with the detectors employed in the methods of the present invention, particularly for use with a mass spectrometers, most preferably a gas phase ion spectrometer. The surface of the substrate includes one or more reactive moieties capable of binding (coupling) polypeptides. Binding moieties can be either mixed or isolated with discrete addressable locations on the substrate. The addressable locations can be arranged in any pattern on the biochip, but are preferably in regular pattern, such as lines, orthogonal arrays, or regular curves (e.g., circles). Exemplary biochips and solid substrates are commercially available (e.g., ProteinChip® Array, Ciphergen Biosystems, Fremont, Calif.). In one preferred embodiment, the substrate itself forms part of a mass spectrometry probe.

2. Reactive Surfaces

Regardless of whether the substrate is suitable for use in mass spectroscopy, at least one substrate surface is preferably conditioned in a manner suitable for creating a binding surface for polypeptides. These binding surfaces, termed reactive surfaces, can be formed by mechanical manipulation of the substrate surface, e.g., roughening, or more preferably by chemical derivatization. Suitable derivatized reactive surfaces include antibodies specific for target polypeptides, nucleic acids such as RNA and DNA aptamers that bind polypeptides as described in U.S. Pat. No. 5,270,163 to Gold et al., Ellington and Szostak, “In vitro Selection of RNA Molecules That Bind Specific Ligands,” Nature 346:818-822 (1990); Gold et al., “Diversity of Oligonucleotide Functions,” Annu. Rev. Biochem. 64:763-797 (1995); and Tuerk and Gold, “Systematic Evolution of Ligands by Exponential Enrichment: RNA Ligands to Bacteriophage T4 DNA Polymerase,” Science 249:505-510 (1990), cellular receptors recognizing target polypeptides, chromatographic capture reagents such as phenyl, alkyl and ionic moieties, and the like. Preferred reactive surfaces include antibodies.

The highly specific nature of antibody binding makes antibodies a preferred capture reagent for forming a reactive surface of the present invention. Methods of producing polyclonal and monoclonal antibodies are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature, 256:495-497 (1975). Such techniques include antibody preparation by selection of antibodies from libraries of recombinant antibodies in phage or similar vectors, as well as preparation of polyclonal and monoclonal antibodies by immunizing mammals (see, e.g., Huse et al., Science, 246:1275-1281 (1989); Ward et al., Nature, 341:544-546 (1989)).

Combinatorial libraries provide an excellent means for selecting capture reagents for reactive surfaces. A combinatorial library is a collection of diverse compounds generated by either chemical synthesis or biological synthesis, by combining a number of chemical “building blocks” such as reagents. For example, a linear combinatorial library, such as a polypeptide library, is formed by combining a set of chemical building blocks (amino acids) in every possible way for a given compound length (i.e., the number of amino acids in a polypeptide compound). Millions of compounds can be synthesized through such combinatorial mixing of chemical building blocks. Preferred combinatorial libraries include collections of antibodies where sequence variability is maximized and limited to the variable regions of the immunoglobulin, and to the production and screening of nucleic acid aptamers. Preparation and screening of combinatorial chemical libraries is well known to those of skill in the art.

III. Detecting Polypeptides

Polypeptides are detected using the methods of the present invention by immobilizing the polypeptides on the reactive surfaces of the biochips described above. The immobilized polypeptides may be washed with an aqueous solution to remove material that is not bound by the reactive surface, or optionally washed with a salt and/or detergent solution to remove material that is loosely bound to the reactive surface. Methods for washing reactive surfaces is well known in the art, and one of skill would be capable of arriving at an optimal washing regime through routine experimentation.

Polypeptides immobilized to the biochips of the present invention may be detected by a variety of detection methods as described above including for example, a gas phase ion spectrometry method, an optical method, an electrochemical method, atomic force microscopy and a radio frequency method. In particular, gas phase ion spectrometry methods are preferred.

In a preferred embodiment, the method for detecting the polypeptides is laser desorption/ionization mass spectrometry and, in particular, SELDI. In one embodiment, a matrix material is applied to the mass spectrometer probe to aid desorption and ionization of the polypeptides. In another embodiment, the SELDI probe can comprise bound energy absorbing molecules and capture reagents. Such SEND/SEAC embodiments eliminate the need to add extraneous matrix. SEND is described further above.

In the preferred mass spectrometric method, data generation begins with the detection of ions generated from the polypeptides by an ion detector. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

IV. Data Analysis

Analysis of data generated by mass spectrometry includes transforming the physical parameters of the sample detected by the mass spectrometer into meaningful molecular mass equivalents, typically through the use of a computer. TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are in U.S. Provisional Patent Application Nos. 60/249,835, filed on Nov. 16, 2000, and 60/254,746, filed on Dec. 11, 2000, and U.S. Non-Provisional patent application Ser. No. 09/999,081, filed Nov. 15, 2001, and Ser. No. 10/084,587, filed on Feb. 25, 2002. All of these U.S. Provisional and Non Provisional Patent Applications are herein incorporated by reference in their entirety for all purposes.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

V. Kits

The present invention also provides kits comprising apparatus and reagents to aid in the detection and Quantitation of polypeptides. In one embodiment, a test kit comprises minimal components necessary to practice the present invention. This includes an expression system capable of expressing at least one inhibitory nucleic acid; and at least one affinity mass spectrometry probe. Additional embodiments include instructions to use the kit to detect expression of a polypeptide whose expression is inhibited by the inhibitory nucleic acid, or a second expression system that does not express the inhibitory nucleic acid with optional instructions for comparing expression of a polypeptide in both the first and second expression systems.

Another kit embodiment of the invention includes at least one inhibitory nucleic acid capable of inhibiting expression of at least one expressed protein of a species and at least one affinity mass spectrometry probe capable of binding at least one of the expressed proteins.

Other embodiments of the present invention include kits preferably packaged to prolong the effective shelf life of associated perishable components. Optionally, the kit may further comprise standard or control information or reagents so that the subject polypeptides can be compared with the control information standard to determine, for example, the degree to which polypeptide expression is inhibited and whether the test procedure is working properly.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims. Those of skill in the art will readily recognize a variety of noncritical parameters that could be changed or modified to yield essentially similar results.

What is claimed is:

1. A method comprising:
a. expressing polypeptides in a first translation system, wherein the first translation system comprises at least one inhibitory nucleic acid directed toward at least one mRNA; and
b. measuring expression of at least one polypeptide in the translation system by affinity mass spectometry.

2. The method of claim 1 wherein the translation system is an in vitro translation system.

3. The method of claim 1 wherein the translation system is an in vivo translation system.

4. The method of claim 1 wherein the translation system is in contact with an affinity surface of an affinity mass spectrometry probe, whereby the affinity surface captures polypeptides expressed by the translation system in situ.

5. The method of claim 1 wherein the inhibitory nucleic acid is selected from an antisense nucleic acid, a ribozyme and an siRNA.

6. The method of claim 5 wherein the inhibitory nucleic acid is an siRNA comprising between 18 and 25 paired bases.

7. The method of claim 1 wherein the expression of at least one polypeptide is inhibited by the at least one inhibitory nucleic acid.

8. The method of claim 1 wherein the at least one inhibitory nucleic acid inhibits expression of a cell receptor selected from a nuclear receptor, a cytoplasmic receptor and a cell surface receptor.

9. The method of claim 8 wherein at least one of the polypeptides is a ligand for the cell receptor.

10. The method of claim 1 wherein the mRNA encodes a component of a signaling pathway.

11. The method of claim 9 wherein at least one of the polypeptides is a component of the pathway.

12. The method of claim 1 wherein the mRNA encodes a kinase or a phosphatase.

13. The method of claim 12 wherein at least one of the polypeptides is a biochemical substrate for the kinase or phosphatase.

14. The method of claim 1 wherein the mRNA encodes a protease.

15. The method of claim 14 wherein at least one of the polypeptides is a biochemical substrate for the protease.

16. The method of claim 1 wherein at least one polypeptide is a polypeptide that interacts with a polypeptide encoded by the mRNA.

17. The method of claim 1 wherein affinity mass spectrometry comprises capturing the at least one polypeptide on a mass spectrometry probe comprising a surface having a chromatographic capture reagent attached thereto.

18. The method of claim 1 wherein affinity mass spectrometry comprises capturing the at least one polypeptide on a mass spectrometry probe comprising a surface having a biospecific capture reagent attached thereto, wherein the biospecific capture reagent binds the polypeptide.

19. The method of claim 1 wherein affinity mass spectrometry further comprises SEND.

20. The method of claim 1 further comprising:
c. expressing second polypeptides in a second translation system, wherein the expression system does not comprise the at least one siRNA;
d. measuring expression of at least one of the second polypeptides in the expression system by affinity mass spectrometry; and,
e. comparing expression of the polypeptides in the first and second expression systems.

21. The method of claim 20 comprising:
i. performing steps (a) and (b) on a plurality of first expression systems and measuring a plurality of first polypeptides;
ii. performing steps (c) and (d) on a plurality of second expression systems measuring a plurality of second polypeptides; and
iii. wherein comparing comprises using the measurements as a learning set to train a learning algorithm, thereby generating a classification model, wherein the classification model uses measurement of at least one polypeptide to classify an unknown sample as belonging to the first or second expression system.

22. A kit comprising:
a. A first expression system capable of expressing at least one inhibitory nucleic acid; and
b. At least one affinity mass spectrometry probe.

23. The kit of claim 22 further comprising instructions to use the kit to detect expression of a polypeptide whose expression is inhibited by the inhibitory nucleic acid.

24. The kit of claim 22 further comprising a second expression system that does not express the inhibitory nucleic acid.

25. The kit of claim 24 further comprising instructions to compare expression of a polypeptide in both the first and second expression systems.

26. A kit comprising:
a. at least one inhibitory nucleic acid capable of inhibiting expression of at least one expressed protein of a species; and,
b. at least one affinity mass spectrometry probe capable of binding at least one of the expressed proteins.
